(19) United States
(12) Patent Application Publication
(10) Pub. No.: US 2025/0312397 A1
(43) Pub. Date: Oct. 9, 2025

(54) FEED FOR INCREASING INTRAMUSCULAR FAT DEPOSITION IN BEEF CATTLE AND PREPARATION METHOD THEREOF

(71) Applicant: Institute of Animal Science and Veterinary Medicine, Shandong Academy of Agricultural Sciences, Jinan (CN)

(72) Inventors: Xianglun Zhang, Jinan (CN); Hongbo Zhao, Jinan (CN); Hongjie Hu, Jinan (CN); Jia You, Jinan (CN); Xiangxing Chen, Jinan (CN); Xiaomu Liu, Jinan (CN); Kaixi Ji, Jinan (CN); Qingkai Sheng, Jinan (CN); Chuanhao Li, Jinan (CN)

(73) Assignee: Institute of Animal Science and Veterinary Medicine, Shandong Academy of Agricultural Sciences, Jinan, SD (CN)

(21) Appl. No.: 19/246,504

(22) Filed: Jun. 23, 2025

(30) Foreign Application Priority Data
Jun. 25, 2024 (CN) 202410827290.0

(51) Int. Cl. A61K 35/747; A23K 10/18; A23K 10/38; A23K 20/105; A23K 20/111; A23K 20/142; A23K 20/20; A23K 50/10; A61K 9/00; A61K 35/00; A61K 36/064; A61K 47/46; A61P 3/02

(52) U.S. Cl. CPC A61K 35/747; A23K 10/18; A23K 10/38; A23K 20/105; A23K 20/111; A23K 20/142; A23K 20/30; A23K 50/10; A61K 9/0056; A61K 36/064; A61K 47/46; A61P 3/02; A23V 2400/125; A61K 2035/115

(57) ABSTRACT

The present invention discloses a feed for increasing intramuscular fat (IMF) deposition in beef cattle and a preparation method thereof, which belongs to the field of animal husbandry feed technology. The present invention promotes the IMF deposition of cattle and improves the marbling grade of beef through the compound feed.

### TECHNICAL FIELD

The present invention belongs to the field of animal husbandry feed technology, particularly a feed for increasing intramuscular fat (IMF) deposition in beef cattle and a preparation method thereof.

### BACKGROUND

Fat is one of the important components of the animal organism, and fat deposition is a very complex biological process that involves the metabolic pathways of multiple nutrient factors in the animal organism, thus determining that fat deposition is affected by the content of multiple nutrient factors in the organism. Fat is divided into visceral fat, subcutaneous fat, intermuscular fat and intramuscular fat (IMF). The content of various fats is an important economic trait in livestock production, particularly IMF content is positively correlated with various sensory indicators, such as juiciness, shear force, tenderness, and flavor of meat. Requirements of consumers for the quality of livestock products such as beef are gradually increasing along with the continuous improvement of consumption and living standards. The content of fat in skeletal muscle, also known as marbling fat, is one of the most important factors determining beef quality. When the IMF content is appropriate, the quality of beef is considered to be high, and the flavor of beef is fit for consumption. However, excessive deposition of non-intramuscular fat (e.g., subcutaneous or visceral fat) reduces cattle feed intake and negatively impacts carcass economic value. Therefore, the regulation of IMF content has become a hot topic in research on improving beef quality.

Current research has found that nutritional factors that may affect IMF deposition; these nutritional factors include fat metabolism, fat digestion and absorption, the effectiveness of glucose and starch, the ratio of roughage to concentrate, the energy and protein levels of the diet, and the levels of vitamins A, D, and C. However, the issue of how to increase IMF deposition in beef cattle by formulating the nutrient composition of the diets fed remains unresolved.

### SUMMARY

In order to solve the above technical problems, the present invention proposes a feed for increasing intramuscular fat (IMF) deposition in beef cattle and a preparation method thereof, through the adjustment of the composition components of the compound feed. This invention will efficiently promote the IMF deposition and enhance the marbling grade of beef.

To achieve the above objective, the present invention provides a feed for increasing IMF deposition in beef cattle, including the following components in parts by weight:

36-40 parts of corn, 12-18 parts of sunflower meal, 6.28-14.96 parts of distiller's grains, 5-10 parts of wheat bran, 5-8 parts of soybean meal, 7-9 parts of flaxseed meal, 2-4 parts of conjugated linoleic acid, 6-10 parts of compound microbial agent, 0.75-1 parts of Radix Isatidis, 0.75-1 parts of Radix Paeoniae Alba, 0.82-1.44 parts of functional additive, and 0.4-0.6 parts of premix.

Preferably, the compound microbial agent includes Lactobacillus casei and/or Saccharomyces cerevisiae.

Further preferably, a mass ratio of Lactobacillus casei to Saccharomyces cerevisiae in the compound microbial agent is 1:1.

Preferably, the functional additive includes the following components in parts by weight: 0.02-0.04 parts of perilla seed, 0.05-0.15 parts of seabuckthorn flavonoid, 0.05-0.15 parts of betaine, 0.3-0.5 parts of glutamine, and 0.4-0.6 parts of daidzein.

Preferably, the premix is prepared by mixing 5000IU of VD3, 60 mg of Fe, 6 mg of Cu, 80 mg of nano zinc oxide, 80 mg of Mn, 0.34 mg of I, and 0.15 mg of Se.

Preferably, the feed includes the following components in parts by weight: 38 parts of corn, 15 parts of sunflower meal, 10.62 parts of distiller's grains, 7.25 parts of wheat bran, 7 parts of soybean meal, 8 parts of flaxseed meal, 3 parts of conjugated linoleic acid, 8 parts of compound microbial agent, 0.75 parts of Radix Isatidis, 0.75 parts of Radix Paeoniae Alba, 1.13 parts of functional additive, and 0.5 parts of premix.

Further preferably, the functional additive includes the following components in parts by weight: 0.03 parts of perilla seed, 0.1 parts of seabuckthorn flavonoid, 0.1 parts of betaine, 0.4 parts of glutamine, and 0.5 parts of daidzein.

The present invention also provides a preparation method for the feed, including the following steps:
(1) obtaining the functional additive by mixing perilla seed, seabuckthorn flavonoid, betaine, glutamine and daidzein;
(2) obtaining a basic feed mixture by mixing corn, sunflower meal, distiller's grains, wheat bran, soybean meal, flaxseed meal and conjugated linoleic acid;
(3) obtaining the feed by mixing the basic feed mixture, the functional additive, the composite microbial agent, the Radix Isatidis, the Radix Paeoniae Alba and the premix.

Compared with the prior art, the present invention has the following advantages and technical effects:
the present invention provides a feed, and the feed is prepared by mixing the basic feed mixture of corn, sunflower meal, distiller's grains, wheat bran, soybean meal, flaxseed meal and conjugated linoleic acid, the functional additive prepared from perilla seed, seabuckthorn flavonoid, betaine, glutamine and daidzein, the composite microbial agent, the Radix Isatidis, the Radix Paeoniae Alba and the premix. The protein content of the feed ingredients has been adjusted to an appropriate level by regulating the proportions of various components in the basic feed, and the fatty acid composition has been improved through the addition of functional additives, thereby moderately increasing IMF content. The addition of composite microbial agents, Radix Isatidis, the Radix Paeoniae Alba enhances the feed digestion capacity of cattle and promotes IMF deposition under high-temperature conditions.

### DETAILED DESCRIPTION OF THE EMBODIMENTS

The following is a detailed description of various exemplary embodiments of the present invention. This detailed description should not be considered as limiting the scope of the present invention, but should be understood as a more detailed description of certain aspects, features, and embodiments of the present invention.

It should be understood that the terms used in the present invention are merely for describing specific embodiments and are not intended to limit the scope of the present invention. Additionally, the numerical ranges disclosed in the present invention should be understood to specifically disclose each intermediate value between the upper and lower limits of the range. Any intermediate value within any stated value or range, as well as any other intermediate value within the stated range, is also included within the scope of the present invention. The upper and lower limits of these smaller ranges may be independently included or excluded from the range.

Unless otherwise noted, all technical and scientific terms used herein have the same meanings as those commonly understood by those skilled in the art described in this invention. Although the present invention describes preferred methods and materials, any methods and materials similar or equivalent to those described herein may also be used in the practice or testing of the present invention. All references cited in this specification are incorporated by reference to disclose and describe the methods and/or materials related to such references. In the event of any conflict with any incorporated reference, the content of this specification shall prevail.

Without departing from the scope or spirit of the present invention, various improvements and changes may be made to the specific embodiments described in the specification of the present invention, which are obvious to those skilled in the art. Other embodiments obtained from the specification of the present invention are obvious to those skilled in the art. The specification and embodiments of the present invention are merely illustrative.

The terms “include,” “comprise,” “have,” “contain,” etc. used in this document are open-ended terms, which means that they include but are not limited to a given component.

Unless otherwise specified, the “parts” referred to in the present invention are calculated by parts by weight.

### Embodiment 1

(1) the functional additive was obtained by mixing 0.03 parts of perilla seed, 0.1 parts of seabuckthorn flavonoid, 0.1 parts of betaine, 0.4 parts of glutamine and 0.5 parts of daidzein;
(2) the basic feed mixture was obtained by mixing 38 parts of corn, 15 parts of sunflower meal, 10.62 parts of distiller's grains, 7.25 parts of wheat bran, 7 parts of soybean meal, 8 parts of flaxseed meal and 3 parts of conjugated linoleic acid;
(3) the composite microbial agent was obtained by mixing Lactobacillus casei freeze-dried powder and Saccharomyces cerevisiae freeze-dried powder at a mass ratio of 1:1;
(4) the feed was obtained by mixing the prepared basic feed mixture, 1.13 parts of the functional additive, 8 parts of the composite microbial agent, 0.75 parts of Radix Isatidis, 0.75 parts of Radix Paeoniae Alba, and 0.5 parts of premix (5000IU of VD3, 60 mg of Fe, 6 mg of Cu, 80 mg of nano zinc oxide, 80 mg of Mn, 0.34 mg of I, and 0.15 mg of Se).

### Embodiment 2

(1) the functional additive was obtained by mixing 0.02 parts of perilla seed, 0.05 parts of seabuckthorn flavonoid, 0.15 parts of betaine, 0.5 parts of glutamine and 0.4 parts of daidzein;
(2) the basic feed mixture was obtained by mixing 36 parts of corn, 12 parts of sunflower meal, 14.96 parts of distiller's grains, 5 parts of wheat bran, 5 parts of soybean meal, 9 parts of flaxseed meal and 2 parts of conjugated linoleic acid;
(3) the composite microbial agent was obtained by mixing Lactobacillus casei freeze-dried powder and Saccharomyces cerevisiae freeze-dried powder at a mass ratio of 1:1;
(4) the feed was obtained by mixing the prepared basic feed mixture, 1.12 parts of the functional additive, 6 parts of the composite microbial agent, 0.75 parts of Radix Isatidis, 0.75 parts of Radix Paeoniae Alba, and 0.42 parts of premix (5000IU of VD3, 60 mg of Fe, 6 mg of Cu, 80 mg of nano zinc oxide, 80 mg of Mn, 0.34 mg of I, and 0.15 mg of Se).

### Embodiment 3

(1) the functional additive was obtained by mixing 0.04 parts of perilla seed, 0.15 parts of seabuckthorn flavonoid, 0.05 parts of betaine, 0.5 parts of glutamine and 0.6 parts of daidzein;
(2) the basic feed mixture was obtained by mixing 40 parts of corn, 18 parts of sunflower meal, 6.28 parts of distiller's grains, 10 parts of wheat bran, 8 parts of soybean meal, 7 parts of flaxseed meal and 4 parts of conjugated linoleic acid;
(3) the composite microbial agent was obtained by mixing Lactobacillus casei freeze-dried powder and Saccharomyces cerevisiae freeze-dried powder at a mass ratio of 1:1;
(4) the feed was obtained by mixing the prepared basic feed mixture, 1.34 parts of the functional additive, 10 parts of the composite microbial agent, 1 part of Radix Isatidis, 1 part of Radix Paeoniae Alba, and 0.6 parts of premix (5000IU of VD3, 60 mg of Fe, 6 mg of Cu, 80 mg of nano zinc oxide, 80 mg of Mn, 0.34 mg of I, and 0.15 mg of Se).

### Embodiment 4

(1) the functional additive was obtained by mixing 0.03 parts of perilla seed, 0.08 parts of seabuckthorn flavonoid, 0.12 parts of betaine, 0.35 parts of glutamine and 0.55 parts of daidzein;
(2) the basic feed mixture was obtained by mixing 37 parts of corn, 17 parts of sunflower meal, 7.27 parts of distiller's grains, 8 parts of wheat bran, 6 parts of soybean meal, 8 parts of flaxseed meal and 3.5 parts of conjugated linoleic acid;
(3) the composite microbial agent was obtained by mixing Lactobacillus casei freeze-dried powder and Saccharomyces cerevisiae freeze-dried powder at a mass ratio of 1:1;
(4) the feed was obtained by mixing the prepared basic feed mixture, 1.13 parts of the functional additive, 7 parts of the composite microbial agent, 0.9 parts of Radix Isatidis, 0.9 parts of Radix Paeoniae Alba, and 0.5 parts of premix (5000IU of VD3, 60 mg of Fe, 6 mg of Cu, 80 mg of nano zinc oxide, 80 mg of Mn, 0.34 mg of I, and 0.15 mg of Se).

### Embodiment 5

(1) the functional additive was obtained by mixing 0.03 parts of perilla seed, 0.12 parts of seabuckthorn flavonoid, 0.08 parts of betaine, 0.45 parts of glutamine and 0.45 parts of daidzein;
(2) the basic feed mixture was obtained by mixing 39 parts of corn, 14 parts of sunflower meal, 9.27 parts of distiller's grains, 6 parts of wheat bran, 7 parts of soybean meal, 8 parts of flaxseed meal and 2.5 parts of conjugated linoleic acid;
(3) the composite microbial agent was obtained by mixing Lactobacillus casei freeze-dried powder and Saccharomyces cerevisiae freeze-dried powder at a mass ratio of 1:1;
(4) the feed was obtained by mixing the prepared basic feed mixture, 1.13 parts of the functional additive, 9 parts of the composite microbial agent, 0.8 parts of Radix Isatidis, 0.8 parts of Radix Paeoniae Alba, and 0.5 parts of premix (5000IU of VD3, 60 mg of Fe, 6 mg of Cu, 80 mg of nano zinc oxide, 80 mg of Mn, 0.34 mg of I, and 0.15 mg of Se).

### Comparative Embodiment 1

(1) the basic feed mixture was obtained by mixing 38 parts of corn, 15 parts of sunflower meal, 10.62 parts of distiller's grains, 7.25 parts of wheat bran, 7 parts of soybean meal, 8 parts of flaxseed meal and 3 parts of conjugated linoleic acid;
(2) the composite microbial agent was obtained by mixing Lactobacillus casei freeze-dried powder and Saccharomyces cerevisiae freeze-dried powder at a mass ratio of 1:1;
(3) the feed was obtained by mixing the prepared basic feed mixture, 8 parts of the composite microbial agent, 0.75 parts of Radix Isatidis, 0.75 parts of Radix Paeoniae Alba, and 0.5 parts of premix (5000IU of VD3, 60 mg of Fe, 6 mg of Cu, 80 mg of nano zinc oxide, 80 mg of Mn, 0.34 mg of I, and 0.15 mg of Se).

### Comparative Embodiment 2

(1) the functional additive was obtained by mixing 0.03 parts of perilla seed, 0.1 parts of seabuckthorn flavonoid, 0.1 part of betaine, 0.4 parts of glutamine and 0.5 parts of daidzein;
(2) the basic feed mixture was obtained by mixing 38 parts of corn, 15 parts of sunflower meal, 10.62 parts of distiller's grains, 7.25 parts of wheat bran, and 7 parts of soybean meal;
(3) the composite microbial agent was obtained by mixing Lactobacillus casei freeze-dried powder and Saccharomyces cerevisiae freeze-dried powder at a mass ratio of 1:1;
(4) the feed was obtained by mixing the prepared basic feed mixture, 1.13 parts of the functional additive, 8 parts of the composite microbial agent, 0.75 parts of Radix Isatidis, 0.75 parts of Radix Paeoniae Alba, and 0.5 parts of premix (5000IU of VD3, 60 mg of Fe, 6 mg of Cu, 80 mg of nano zinc oxide, 80 mg of Mn, 0.34 mg of I, and 0.15 mg of Se).

### Comparative Embodiment 3

(1) the functional additive was obtained by mixing 0.03 parts of perilla seed, 0.1 parts of seabuckthorn flavonoid, 0.1 parts of betaine, 0.4 parts of glutamine and 0.5 parts of daidzein;
(2) the basic feed mixture was obtained by mixing 38 parts of corn, 15 parts of sunflower meal, 10.62 parts of distiller's grains, 7.25 parts of wheat bran, 7 parts of soybean meal, 8 parts of flaxseed meal and 3 parts of conjugated linoleic acid;
(3) the composite microbial agent was obtained by mixing Lactobacillus casei freeze-dried powder and Saccharomyces cerevisiae freeze-dried powder at a mass ratio of 1:1;
(4) the feed was obtained by mixing the prepared basic feed mixture, 1.13 parts of the functional additive, 8 parts of the composite microbial agent, and 0.5 parts of premix (5000IU of VD3, 60 mg of Fe, 6 mg of Cu, 80 mg of nano zinc oxide, 80 mg of Mn, 0.34 mg of I, and 0.15 mg of Se).

### Experimental Example 1

16-month-old Angus steers with body weight in the range of 400-430 kg were selected and divided into 8 groups, 10 in each group, the fattening cattle fed with the feed prepared by embodiments 1-5 were the experimental group 1-5, and the fattening cattle fed with the feed prepared by the comparative embodiments 1-3 were the control group 1-3. The test period was 120 days, with twice daily feedings on a regular schedule in the morning and evening. The actual amount of feeding feed per day and the remaining amount of feed per day were recorded. During this period, the fattening cattle were free to feed and water.

The initial body weight (IBW) of fattening cattle was measured before the start of the experiment, and the final body weight (FBW) of fattening cattle was measured after the experiment, the average daily gain (ADG) of fattening cattle in each group was calculated according to the measured body weight.

The dry matter content of the supplied feed and the remaining feed was measured every 10 days, and the average daily dry matter intake (ADDMI) of each 10-day period was calculated, the ADDMI of the whole test period was calculated based on the ADDMI of each 10-day period. The feed/gain ratio (F/G) was calculated according to ADDMI and ADG.

TABLE 1
Results of growth performance measurements for fattening cattle in different groups

| | BBW | FBW | ADG | ADDMI | |
|---|---|---|---|---|---|
| Group | (kg) | (kg) | (kg) | (kg) | F/G |
| Experimental | 414.28 ± 15.03 | 539.18 ± 15.57 | 1.04 ± 0.16 | 7.89 ± 0.19 | 7.58 ± 1.02 |
| group 1 | | | | | |
| Experimental | 413.28 ± 16.78 | 523.68 ± 19.45 | 0.92 ± 0.22 | 7.25 ± 0.20 | 7.89 ± 1.15 |
| group 2 | | | | | |
| Experimental | 417.03 ± 16.22 | 534.63 ± 18.15 | 0.98 ± 0.20 | 7.63 ± 0.25 | 7.79 ± 1.08 |
| group 3 | | | | | |
| Experimental | 415.09 ± 15.47 | 529.09 ± 17.61 | 0.95 ± 0.19 | 7.51 ± 0.22 | 7.91 ± 1.11 |
| group 4 | | | | | |
| Experimental | 416.72 ± 14.61 | 533.12 ± 15.85 | 0.97 ± 0.16 | 7.71 ± 0.22 | 7.95 ± 1.04 |
| group 5 | | | | | |
| Control | 415.23 ± 15.68 | 510.03 ± 19.03 | 0.79 ± 0.23 | 6.60 ± 0.35 | 8.35 ± 1.18 |
| group 1 | | | | | |
| Control | 417.06 ± 14.96 | 503.46 ± 14.45 | 0.72 ± 0.15 | 5.86 ± 0.38 | 8.14 ± 1.24 |
| group 2 | | | | | |
| Control | 413.89 ± 16.27 | 515.89 ± 13.31 | 0.85 ± 0.18 | 7.02 ± 0.36 | 8.26 ± 1.27 |
| group 3 | | | | | |

According to the results recorded in Table 1, the ADG of the experimental groups 1-5 fed with the feed described in the present invention is higher than that of the control groups 1-3, and the F/G ratio is significantly reduced, which improves the utilization rate of feed. The fattening cattle in the control groups 1-3 were fed with a higher F/G ratio and less daily weight gain.

### Experimental Example 2

In experimental example 1, after 120 days of feeding, three fattening cattle closest to the average weight were selected from each group. After 24 hours of fasting, the cattle were slaughtered, and samples of the longissimus dorsi muscle were collected to measure the pH, shear force, water-holding capacity, and IMF content of the longissimus dorsi muscle. The pH of the longissimus dorsi muscle was measured with a 3D PH meter. The methods used for the determination of the shear force of the longissimus dorsi muscle and the water-holding force of the longissimus dorsi muscle were conventional techniques. The IMF content of the longissimus dorsi muscle was determined by Soxhlet fat extraction.

TABLE 2
Results of meat quality measurements for
fattening cattle in different groups

| | | Shear | Water-holding | IMF content |
|---|---|---|---|---|
| | | force of the | capacity of the | of the |
| | pH of the | longissimus | longissimus | longissimus |
| | longissimus | dorsi muscle | dorsi muscle | dorsi muscle |
| Group | dorsi muscle | (N) | (%) | (%) |
| Experimental | 4.72 ± 0.12 | 7.21 ± 0.95 | 94.55 ± 1.47 | 4.72 ± 0.12 |
| group 1 | | | | |
| Experimental | 4.74 ± 0.15 | 7.25 ± 0.94 | 95.02 ± 1.56 | 4.68 ± 0.11 |
| group 2 | | | | |
| Experimental | 4.72 ± 0.14 | 7.30 ± 0.97 | 95.43 ± 1.52 | 4.71 ± 0.15 |
| group 3 | | | | |
| Experimental | 4.76 ± 0.12 | 7.28 ± 0.92 | 94.67 ± 1.50 | 4.65 ± 0.12 |
| group 4 | | | | |
| Experimental | 4.75 ± 0.10 | 7.26 ± 0.98 | 94.73 ± 1.46 | 4.75 ± 0.13 |
| group 5 | | | | |
| Control | 4.50 ± 0.24 | 9.23 ± 1.15 | 96.85 ± 1.03 | 4.21 ± 0.18 |
| group 1 | | | | |
| Control | 4.53 ± 0.23 | 9.05 ± 1.14 | 96.14 ± 1.07 | 4.18 ± 0.25 |
| group 2 | | | | |
| Control | 4.61 ± 0.24 | 9.18 ± 1.20 | 95.87 ± 1.07 | 4.47 ± 0.31 |
| group 3 | | | | |

According to the results recorded in Table 2, the experimental groups 1-5 fed with the feed described in the present invention significantly reduced the shear force of the longissimus dorsi muscle and increased the IMF content of the longissimus dorsi muscle.

Experimental example 3 Marbling grades were scored for the fattening cattle slaughtered in experimental example 2: the marbling of the fattening cattle was scored in accordance with Beef quality grading NYT676-2010, the scores were graded from low to high from 1 to 5 points, with three people scoring simultaneously and the average value being taken.

TABLE 3
Evaluation results of marbling grade of fattening beef in different groups

| | Experimental | Experimental | Experimental | Experimental | Experimental | Control | Control | Control |
|---|---|---|---|---|---|---|---|---|
| Group | group 1 | group 2 | group 3 | group 4 | group 5 | group 1 | group 2 | group 3 |
| Score | 4.47 ± 0.15 | 4.35 ± 0.35 | 4.42 ± 0.41 | 4.29 ± 0.36 | 4.33 ± 0.28 | 3.42 ± 0.89 | 3.89 ± 0.74 | 3.85 ± 0.15 |

According to the results recorded in Table 3, it can be seen that the marbling grades of fattening beef in the experimental group fed with the feed described in the present invention are all in the upper-middle grade.

The above embodiments are merely descriptions of preferred embodiments of the present invention and are not intended to limit the scope of the present invention. Any modifications or improvements made by ordinary technicians in the art to the technical solutions of the present invention without departing from the spirit of the present invention should fall within the scope of protection defined by the claims of the present invention.

What is claimed is:

1. A feed for increasing intramuscular fat (IMF) deposition in beef cattle, wherein the feed comprises the following components in parts by weight:
36-40 parts of corn, 12-18 parts of sunflower meal, 6.28-14.96 parts of distiller's grains, 5-10 parts of wheat bran, 5-8 parts of soybean meal, 7-9 parts of flaxseed meal, 2-4 parts of conjugated linoleic acid, 6-10 parts of compound microbial agent, 0.75-1 parts of Radix Isatidis, 0.75-1 parts of Radix Paeoniae Alba, 0.82-1.44 parts of functional additive, and 0.4-0.6 parts of premix.

2. The feed according to claim 1, wherein the compound microbial agent comprises Lactobacillus casei and/or Saccharomyces cerevisiae.

3. The feed according to claim 2, wherein a mass ratio of Lactobacillus casei to Saccharomyces cerevisiae in the compound microbial agent is 1:1.

4. The feed according to claim 1, wherein the functional additive comprises the following components in parts by weight:
0.02-0.04 parts of perilla seed, 0.05-0.15 parts of seabuckthorn flavonoid, 0.05-0.15 parts of betaine, 0.3-0.5 parts of glutamine, and 0.4-0.6 parts of daidzein.

5. The feed according to claim 1, wherein the premix is prepared by mixing 5000IU of VD3, 60 mg of Fe, 6 mg of Cu, 80 mg of nano zinc oxide, 80 mg of Mn, 0.34 mg of I, and 0.15 mg of Se.

6. The feed according to claim 1, wherein the feed comprises the following components in parts by weight:
38 parts of corn, 15 parts of sunflower meal, 10.62 parts of distiller's grains, 7.25 parts of wheat bran, 7 parts of soybean meal, 8 parts of flaxseed meal, 3 parts of conjugated linoleic acid, 8 parts of compound microbial agent, 0.75 parts of Radix Isatidis, 0.75 parts of Radix Paeoniae Alba, 1.13 parts of functional additive, and 0.5 parts of premix.

7. The feed according to claim 4, wherein the functional additive comprises the following components in parts by weight:
0.03 parts of perilla seed, 0.1 parts of seabuckthorn flavonoid, 0.1 parts of betaine, 0.4 parts of glutamine, and 0.5 parts of daidzein.

8. A method of preparing the feed according to claim 7, wherein the method comprises the following steps:
(1) obtaining the functional additive by mixing perilla seed, seabuckthorn flavonoid, betaine, glutamine and daidzein;
(2) obtaining a basic feed mixture by mixing corn, sunflower meal, distiller's grains, wheat bran, soybean meal, flaxseed meal and conjugated linoleic acid; and
(3) obtaining the feed by mixing the basic feed mixture, the functional additive, the composite microbial agent, the Radix Isatidis, the Radix Paeoniae Alba and the premix.
